(19) United States
(12) Patent Application Publication
(10) Pub. No.: US 2005/0149056 A1
(43) Pub. Date: Jul. 7, 2005

(54) IOL injector device and method

(76) Inventor: Brian D. Rathert, St. Petersburg, FL (US)

Correspondence Address: Bausch & Lomb Incorporated, One Bausch & Lomb Place, Rochester, NY, 14604-2701, US

(21) Appl. No.: 10/744,980

(22) Filed: Dec. 22, 2003

(51) Int. Cl. A61F 9/00

(52) U.S. Cl. 606/107

(57) ABSTRACT

Improved closing and locking mechanism for an IOL compressor component of an IOL injector device includes two equally spaced, sequential pairs of catches provided on a wall of the compressor for sequentially engaging with a respective pair of detents formed adjacent the compressor opening of the injector body as the compressor is moved to the closed position.

### BACKGROUND OF THE INVENTION

The present invention relates to ophthalmic surgical devices and methods. More particularly, the present invention relates to a device and method for inserting an intraocular lens (IOL) into an eye.

IOLs are artificial lenses used to replace the natural crystalline lens of the eye when the natural lens has cataracts or is otherwise diseased. IOLs are also sometimes implanted into an eye to correct refractive errors of the eye in which case the natural lens may remain in the eye together with the implanted IOL. The IOL may be placed in either the posterior chamber or anterior chamber of the eye. IOLs come in a variety of configurations and materials. Some common IOL styles include the so-called open-looped haptics which include the three-piece type having an optic and two haptics attached to and extending from the optic; the one-piece type wherein the optic and haptics are integrally formed (e.g., by machining the optic and haptics together from a single block of material); and also the closed looped haptic IOLs. Yet a further style of IOL is called the plate haptic type wherein the haptics are configured as a flat plate extending from opposite sides of the optic. The IOL may be made from a variety of materials or combination of materials such as PMMA, silicone, hydrogels and silicone hydrogels, etc.

Various instruments and methods for implanting the IOL in the eye are known. In one method, the surgeon simply uses surgical forceps having opposing blades which are used to grasp the IOL and insert it through the incision into the eye. While this method is still practiced today, more and more surgeons are using more sophisticated IOL injector devices which offer advantages such as affording the surgeon more control when inserting the IOL into the eye. IOL injector devices have recently been developed with reduced diameter insertion tips which allow for a much smaller incision to be made in the cornea than is possible using forceps alone. Smaller incision sizes (e.g., less than about 3 mm) are preferred over larger incisions (e.g., about 3.2 to 5+mm) since smaller incisions have been attributed to reduced post-surgical healing time and complications such as induced astigmatism.

Since IOLs are very small and delicate articles of manufacture, great care must be taken in their handling. In order for the IOL to fit through the smaller incisions, they need to be folded and/or compressed prior to entering the eye wherein they will assume their original unfolded/uncompressed shape. The IOL injector device must therefore be designed in such a way as to permit the easy passage of the IOL through the device and into the eye, yet at the same time not damage the delicate IOL in any way. Should the IOL be damaged during delivery into the eye, the surgeon will most likely need to extract the damaged IOL from the eye and replace it with a new IOL, a highly undesirable surgical outcome.

Thus, as explained above, the IOL injector device must be designed to permit easy passage of the IOL therethrough. It is equally important that the IOL be expelled from the tip of the IOL injector device and into the eye in a predictable orientation and manner. Should the IOL be expelled from the tip too quickly or in the wrong orientation, the surgeon must further manipulate the IOL in the eye which could result in trauma to the surrounding tissues of the eye. It is therefore highly desirable to have an injector device which allows for precise loading and compression of the IOL into the injector device and which will pass and expel the IOL from the injector device tip and into the eye in a controlled, predictable and repeatable manner.

To ensure controlled expression of the IOL through the tip of the IOL injector device, the IOL must first be loaded and folded and/or compressed (hereinafter collectively referred to as “compress”) in the IOL injector device. The loading and compressing of the IOL into the injector device is therefore a precise and very important step in the process. Incorrect loading and/or compressing of an IOL into the injector device is oftentimes cited as the reason for a failed IOL delivery sequence. Many IOL injector devices on the market today require the IOL to be loaded and compressed in the injector at the time of surgery by the attending nurse and/or surgeon. Due to the delicate nature of the IOL, there is a risk that the nurse and/or surgeon will inadvertently damage the IOL during the loading and compressing steps resulting in a failed implantation. It is therefore important that the injector device not have any parts which could inadvertently harm the IOL while being loaded and compressed. In addition, visualization of the IOL loading and compression area during loading, compression and delivery enables the surgeon or nurse to ascertain the condition of the IOL. It is therefore furthermore desirable to have an IOL injector device which provides a loading area and mechanism for compressing the IOL within the injector device which allows good visualization of the IOL loading and compressing area.

### SUMMARY OF THE INVENTION

The present invention provides an injector device which enables the reliable loading, compressing and delivery of an IOL therethrough. More particularly, the invention provides an injector device for delivering an IOL into an eye wherein the injector device includes a compressor mechanism for compressing the IOL, the compressor including one or more catch and detents provided on facing surfaces thereof so as to prevent the compressor from inadvertently releasing from the injector device. The catch and detent pairs are positioned laterally of the IOL loading chamber such that good visualization and protection of the IOL in the loading chamber is maintained throughout the IOL compression step. In an advantageous embodiment, two pairs of catch and detent pairs are provided in spaced relation to enable sequential advancement and locking of the compressor with respect to the IOL loading chamber.

### BRIEF DESCRIPTION OF THE DRAWINGS

FIG. 1 is a perspective view of an embodiment of the injector device with an IOL loaded therein ready for compression and delivery into an eye;

FIG. 2 is a partial perspective view of the injector device of FIG. 1 showing the compressor in the fully closed position;

FIG. 3 is the view of FIG. 2 showing the injector absent the compressor component;

FIG. 4 is a perspective view of the compressor component;

FIG. 5 is the view of FIG. 2 showing an IOL being expressed at the tip of the injector;

FIGS. 6A-C are cross-sectional views of the injector showing sequential advancement and closing of the compressor component and resultant compression of the IOL situated therein; and

FIG. 7 is a top plan view of the distal portion of the injector device showing the compressor component in the open position and the leading and trailing haptics engaged with the haptic puller and plunger tip in the intended manner, respectively.

### DETAILED DESCRIPTION

Referring now to the drawing, there is seen in FIG. 1 an injector device denoted generally by the reference numeral 10. Injector device 10 is used to compress and deliver an IOL 12 into an eye (not shown). The injector 10 is generally of the construction shown and described in commonly assigned U.S. Pat. No. 5,944,725, the entire disclosure of which is incorporated by reference. The present invention provides a different locking position for the compressor denoted 40 in the '725 patent and numeral 20 in the instant application figures. The principle advantage of the present invention over the '725 device is that the locking mechanism is no longer positioned adjacent the loaded IOL when the compressor is in the fully closed position. This feature eliminates the possibility of the locking mechanism coming into contact with the loaded IOL which could damage the IOL. Further, by having the locking mechanism positioned laterally of the longitudinal passageway and particularly the IOL loading area, visualization of the loaded IOL is improved.

Injector device 10 comprises an injector body 11 having a longitudinal passageway 13 extending between opposite proximal and distal ends 14, 16, respectively, and through which a plunger 15 telescopes at proximal end 14. Plunger 15 is provided with a tip 15a (FIG. 7) for engaging and advancing the IOL 12 through the device to exit at injector distal tip 17 (see FIG. 5). This is accomplished by manually advancing the plunger in the manner of a syringe with the thumb pushing against thumb press 15b and the fingers engaging the finger plate 24 of the injector body. Finger plate 24 is preferably configured with a straight edge 24a as shown (FIG. 1) for resting device 10 on a flat surface.

The passageway 13 may assume any desired cross-sectional shape such as a rounded, rectangular shape as shown. It is preferred that unintentional rotation of the plunger with respect to the injector body is prevented. For example, the longitudinal passageway and plunger shaft may be made non-circular in cross-section as shown, or other features may be used to prevent rotation as desired.

The longitudinal passageway tapers inwardly at distal tip 17 so that the IOL 12 is gradually compressed to a very small cross-section (e.g., less than or about 3 mm) as it exits the device at tip 17.

It is understood that the overall configuration of the injector body 11 may vary from that shown and described herein. It is furthermore understood that the components of the injector device may be made of any suitable material and may be wholly or partly opaque, transparent or translucent to better visualize the IOL within the injector device and the IOL delivery sequence. Examples of such materials include, but are not limited to, polypropylene, polycarbonate, polysulfone, ALTEM (by Dupont), and PFA.

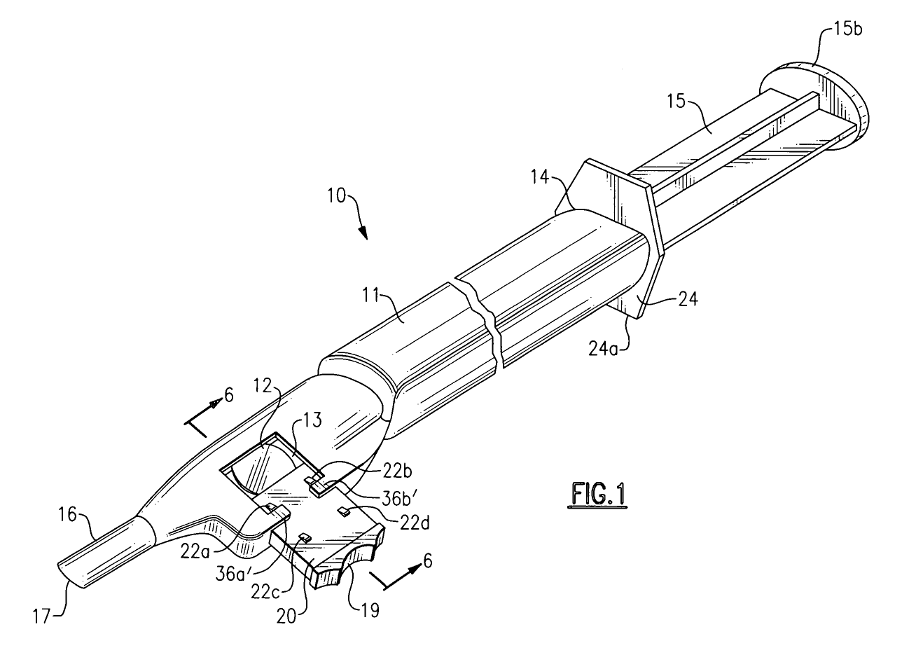

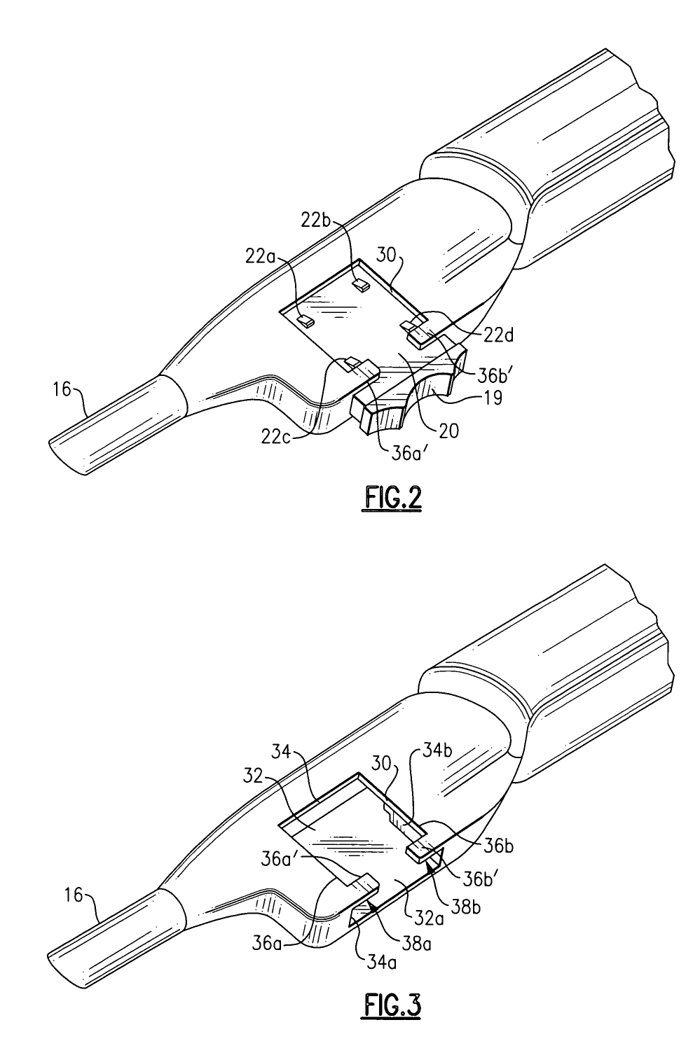

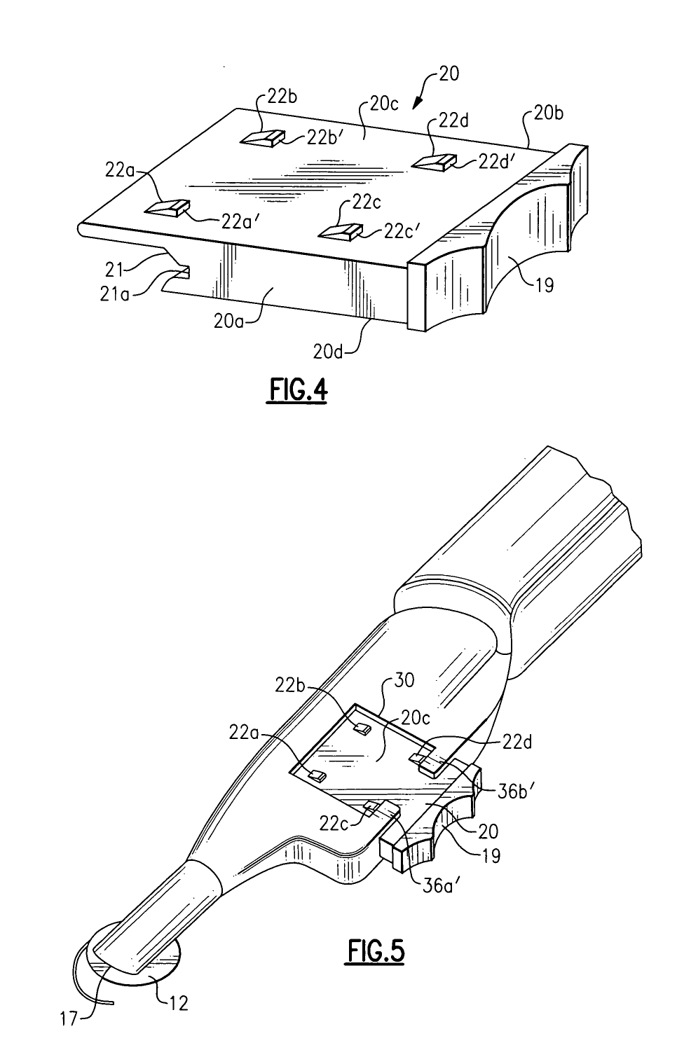

Attention is now turned to the loading and compression sequence of IOL 12 within the injector 10. Injector body 11 includes an opening 30 in the side wall thereof the opening 30 being defined by bottom wall 32 and side wall 34 of the injector body (see FIG. 3). Bottom wall 32 extends laterally outward of the injector body 11 to define a shelf segment 32a having opposite side walls 34a, 34b and respective top wall segments 36a, 36b together defining respective longitudinal grooves 38a, 38b for slidably receiving opposite side walls 20a, 20b of compressor 20, respectively (see FIG. 4). Top wall segments 36a, 36b include extension segments 36a′, 36b′ defining respective detents 36c, 36d for engaging with the catches 22a-d provided on compressor top wall 20c as described more fully below.

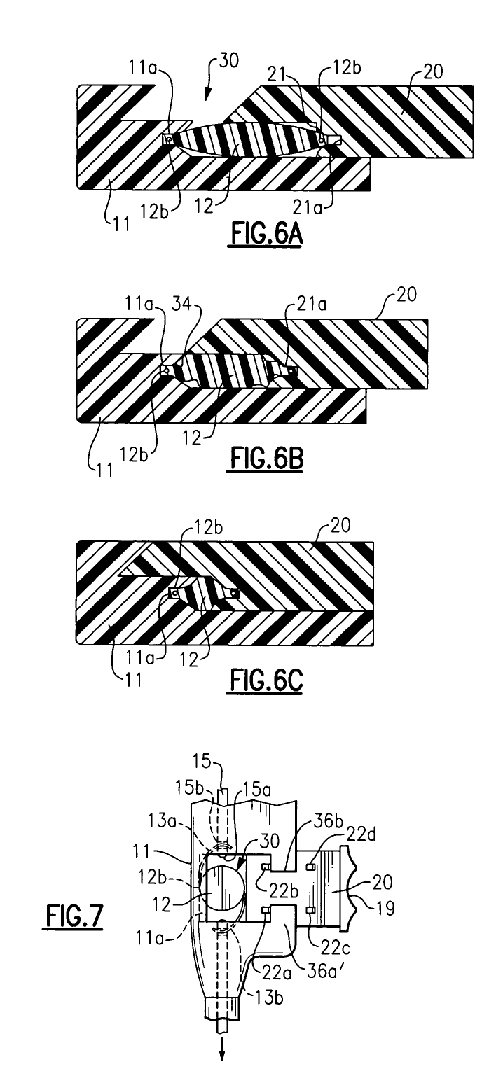

Injector body opening 30 is sized to permit placement of an unstressed IOL 12 within injector body 11 using surgical forceps, for example. The IOL 12 is positioned in opening 30 with the optic edge 12b thereof engaged in longitudinal groove 11a extending along the side wall 34 (see also FIGS. 6A-C and 7). When the IOL 12 is initially placed in opening 30, compressor 20 is in the completely open position shown in FIGS. 1, 6a and 7. When in the fully open position, compressor 20 has been advanced into opening 30 only up to the point where the first set of catches 22a, 22b extend past the detents 36a′, 36b′ whereby movement of compressor 20 in the opposite direction (away from injector body 11) is prohibited due to the right angle faces 22a′, 22b′ thereof (FIG. 4) abutting against detents 36a′, 36b′, respectively. In this regard, it is noted that the shape of the catches is that of a wedge to provide a relatively easy sliding engagement with the detents, although other configurations are possible and within the scope of the invention. Additionally, the catches and detents may be reversed in position, i.e., the catches may be placed on the injector body and the detents may be provided on the compressor. Further, the placement of the detents or catches may be either on the bottom wall 20d or top wall 20c of the compressor 20. If placed on the bottom wall 20d, the corresponding catches/detents would be located adjacent the shelf segment 32a of the injector body 11. The detents may further be of any desired configuration including, for example, a recess, with the only requirement being that the catch and respective detent engage one another so as to prevent the compressor from moving in the reverse direction.

FIGS. 6A-C show the sequential advancement of compressor 20 and resultant compression of IOL 12. In particular, it is seen in FIG. 6A that compressor 20 may be advanced toward the closed position by manually pressing against the finger push 19 located opposite leading surface 21. As compressor 20 is advanced in the direction of injector body side wall 34, the opposite edge 12b′ of the optic 12 becomes engaged by the longitudinal groove 21a formed in the leading surface 21 of compressor 20. Further advancement of compressor 20 as shown in FIG. 6B initiates compression of IOL 12 as it becomes squeezed between opposing grooves 11a and 21a. Upon fully closing compressor 20, the IOL 12 is compressed to the condition shown in FIG. 6C and the second set of catches 22c, 22d have passed and engaged detents 36a′, 36b′, respectively. At this point, compressor 20 is essentially locked in the fully closed position, again due to the right angle faces 22c′, 22d′ abutting shoulders 36c, 36d, respectively. In the situation of delivering an IOL having two looped haptics 13a and 13b as seen in FIG. 7, the leading haptic 13b is engaged in a groove 19a of a haptic puller 19 which the user pulls out of the tip 17 prior to IOL delivery. This action pulls the leading haptic 13b into the tip 17 to prevent the haptic from “bunching” upon itself as the IOL 12 is advanced by the plunger 15. This haptic puller configuration and operation is described in detail in commonly assigned U.S. Pat. No. 5,944,725. Once the haptic puller has been removed from te tip 17, the user may proceed to inject the IOL 12 into the patient's eye by inserting tip 17 into the eye and advancing plunger 15 until the IOL 12 is expressed from tip 17 into the eye. It will further be noted that the plunger tip 15a includes a relief 15b wherein the trailing haptic 13a resides when the IOL 12 is initially placed in opening 30. This ensures the plunger tip will bypass the trailing haptic and engage the optic edge 12b to push the IOL through and out tip 17 in the intended manner. The expression of the IOL 12 from the injector device is controlled and provides a “planar” delivery principally owing to the IOL optic being guided through the device by the opposing longitudinal grooves 11a, 21a, and 11b.

As stated above, the detents 22a-d may take on a variety of configurations so long as they serve the purpose of inhibiting the compressor 20 from inadvertently opening or releasing from the injector body 11. Furthermore, the total number of detents provided may vary from one or more as desired, although two equally spaced, sequential pairs are preferred as shown in the drawing to enhance overall stability between the compressor and injector body.

It may thus be realized that the present invention provides an injector device and method employing a compressor mechanism that may be provided in a variety of embodiments. The present invention is therefore not to be limited by the embodiments shown and described herein but is to be defined by the claims which follow.

1. An injector device for injecting an IOL into an eye, said injector device comprising:
a) an injector body having a longitudinal passageway and an opening formed in a side wall of said body for placement of an IOL therein;
b) a compressor having a leading surface, a finger push surface, a top wall, a bottom wall and opposite side walls, said compressor adapted to be received in said opening and movable between opened and closed positions with respect to said injector body;
c) at least one catch formed on one of said compressor top wall and bottom wall;
d) at least one detent formed on said injector body adjacent said opening and laterally of said longitudinal passageway,
whereby said compressor may be moved from said open position to said closed position to compress the IOL in the injector body with said at least one catch engaging said at least one detent to prevent said compressor from moving in the reverse direction.

2. An injector device for injecting an IOL into an eye, said injector device comprising:
a) an injector body having a longitudinal passageway and an opening formed in a side wall of said body for placement of an IOL therein;
b) a compressor having a leading surface, a finger push surface, a top wall, a bottom wall and opposite side walls, said compressor adapted to be received in said opening and movable between a open and closed positions with respect to said injector body;
c) at least one detent formed on one of said compressor top wall and bottom wall;
d) at least one catch formed on said injector body adjacent said opening and laterally of said longitudinal passageway,
whereby said compressor may be moved from said open position to said closed position to compress the IOL in the injector body with said at least one catch engaging said at least one detent to prevent said compressor from moving in the reverse direction.

3. The device of claim 1, wherein two, laterally spaced detents are formed on one of said compressor top wall and bottom wall.

4. The device of claim 1, wherein two pairs of detents are formed on one of said compressor top wall and bottom wall.

5. The device of claim 1, wherein said IOL has a leading haptic and a trailing haptic, and further comprising a haptic puller for placing in said distal tip of said device and engaging with the leading haptic of said IOL, said haptic puller being removable from said device whereupon said leading haptic is straightened within said distal tip.

6. The device of claim 1 wherein two of said catches are formed on said injector body by a respective pair of top wall extensions formed adjacent said opening.

7. The device of claim 1 wherein said detents are wedge shaped and slidingly engage with said catches, respectively.
